(19) United States
(12) Patent Application Publication
(10) Pub. No.: US 2018/0020976 A1
(43) Pub. Date: Jan. 25, 2018

(54) ORALLY INSERTED PROBE AND METHOD FOR MEASURING VITAL SIGNS

(71) Applicant: Respidx LTD, Tel Aviv (IL)

(72) Inventor: Aldar Yossi, Tel Aviv (IL)

(73) Assignee: Respidx LTD, Tel Aviv (IL)

(21) Appl. No.: 15/547,276

(22) PCT Filed: Jan. 28, 2016
(86) PCT No.: PCT/IL2016/050090

§ 371 Date: Jul. 28, 2017

Related U.S. Application Data
(60) Provisional application No. 62/108,541, filed on Jan. 28, 2015.

(51) Int. Cl. A61B 5/00; A61B 5/0205; A61B 5/1455

(52) U.S. Cl. CPC A61B 5/682; A61B 5/14552; A61B 5/02055; A61B 5/7405; A61B 5/0022; A61B 5/0816

(57) ABSTRACT

An orally inserted probe and method for measuring vital signs of a mammalian subject is disclosed. Respiratory sensors and a pulse oximetry sensor can measure respiratory rate, heart rate, and oxygen saturation. The sensors are kept in position by a mouthpiece held in the mouth, and by other features of the probe described herein. The mouthpiece can also contain a body temperature sensor. The probe is easy to use, even by an untrained caregiver. A method of measuring frequency of physical changes near a mammalian subject's nose or nostrils can be used to measure respiratory rate. A method of sharing vital signs measurement data with an external device and utilization of the data by medical personnel and caregivers is also disclosed.

### FIELD OF THE INVENTION

The present invention generally pertains to an orally inserted probe and a method for measuring vital signs.

### BACKGROUND TO THE INVENTION

Measuring and monitoring vital signs, such as respiratory rate, heart rate, oxygen saturation, and body temperature, is a key factor in diagnosis and management of numerous infectious diseases.

Monitoring respiratory rate is especially important when treating respiratory conditions such as pneumonia, in which a reduction in lung capacity causes a mammalian subject to breathe more rapidly in order to increase body oxygenation. In many cases, detecting a rapid respiratory rate can be the sole indicator of this condition.

Measuring respiratory rate is often complicated by the mammalian subject's breathing through their nose or their mouth, or alternating between the two. A reading of respiratory rate is typically performed by medical personnel such as a trained doctor or nurse, using a stethoscope and a clock. This method requires a skill level generally not present in the home environment or in the ambulatory environment.

It is also well known that many medical conditions, such as pneumonia, are characterized by low oxygen saturation. Furthermore, many medical conditions are accompanied by too rapid a heart rate (tachycardia) or too slow a heart rate (bradycardia). Thus, regular pulse oximetry readings of oxygen saturation and heart rate also provide a useful tool for screening for disease.

It is also well known that many medical conditions are associated with increased body temperature. This is particularly the case with infectious diseases such as pneumonia. Thus regular measurements of body temperature are also a useful screening tool for these diseases.

There is therefore a long-felt but unmet need for a means for unskilled persons to measure vital signs such as respiratory rate, temperature, oxygen saturation and heart rate in situations where medical personnel are not present; for example, in home care or ambulatory care. There is also such a need in developing countries, where medical personnel typically undergo far less training than their developed-nation counterparts.

### SUMMARY OF THE INVENTION

It is therefore the objective of the present invention to enable simple determination of vital signs by an untrained caregiver.

It is a further objective of the invention to provide a small, easy-to-use device to achieve said objective.

It is a further objective of the invention to ensure that the device is ergonomic, anatomically conformed, and safe to use, even for an infant mammalian subject.

It is a still further objective of the invention to measure at least two vital signs simultaneously, for example temperature together with the mammalian subject's respiratory rate, or temperature together with the vital signs measurable via pulse oximetry (i.e. heart-rate and oxygen saturation).

It is a still further objective of the invention to enable data storage of the measured vital signs data.

It is a still further objective of the invention to communicate the measured vital signs data to one or more devices located nearby and/or remotely.

It is a still further objective of the invention to provide a probe [100] configured for measurement of vital signs of a mammalian subject, the vital signs comprising at least one of respiratory rate, heart rate, and oxygen saturation of a mammalian subject, comprising
a. a mouthpiece [10] configured for insertion into a mouth of said mammalian subject; and
b. one or more sensors configured to perform the measurement of vital signs, wherein said one or more sensors are each disposed in a position suitable for measurement when the mouthpiece is inserted in the mouth of the mammalian subject.

It is a still further objective of the invention to provide the probe [100] configured for measurement of vital signs mentioned above, further wherein
a. the vital signs include respiratory rate and the one or more sensors comprise one or more respiratory sensors; and
b. the position suitable for measurement is in proximity to the mammalian subject's nose, thereby wherein said one or more respiratory sensors are disposed in proximity to the nose of the mammalian subject at such time as the mouthpiece is in the mouth of the mammalian subject,
wherein by means of a waveform of physical changes measurable by the at least one respiratory sensor the respiratory rate of the mammalian subject is determinable.

It is a still further objective of the invention to provide the probe [100] configured for measurement of vital signs mentioned above, wherein
a. the vital signs include at least one of heart rate and oxygen saturation and the one or more sensors comprise a reflective pulse oximetry sensor; and
b. the position suitable for measurement is adjacent to and facing a lip of the mammalian subject, thereby wherein the reflective pulse oximetry sensor is disposed adjacent to and facing the lip of the mammalian subject at such time as the mouthpiece is in the mouth of the mammalian subject.

It is a still further objective of the invention to provide a probe [100] configured for measurement of vital signs mentioned above, wherein
a. the vital signs further include body temperature; and
b. the mouthpiece [10] further comprises a body temperature sensor [24], disposed in the mouthpiece [10] and configured for measurement of body temperature.

It is a still further objective of the invention to provide the probe [100] configured to measure respiratory rate mentioned above, wherein
a. the at least one respiratory sensor comprises two respiratory sensors;
b. the two respiratory sensors are in proximity to two nostrils of the mammalian subject, one sensor in proximity with each nostril.

It is a still further objective of the invention to provide a probe [100] configured to measure respiratory rate mentioned above, wherein the at least one respiratory sensor [16] comprises one or more temperature sensors.

It is a still further objective of the invention to provide the probe [100] configured to measure respiratory rate and comprising one or more temperature sensors mentioned above, wherein the one or more temperature sensors are selected from a group including thermocouples thermopiles, and thermistors.

It is a still further objective of the invention to provide a probe [100] configured to measure respiratory rate mentioned above, further comprising a processor [36] configured to perform an analysis of the waveform [FIG. 4] generated by the at least one respiratory sensor [16], the processor [36] configured to determine the respiratory rate.

It is a still further objective of the invention to provide the probe [100] configured to measure respiratory rate and comprising a processor mentioned above, wherein the processor [36] is further configured to perform the analysis using a Fourier transform of the waveform.

It is a still further objective of the invention to provide a probe [100] configured for measurement of vital signs mentioned above, wherein a cross section of at least a portion of the mouthpiece [10] is horizontally elongated.

It is a still further objective of the invention to provide a probe [100] configured for measurement of vital signs mentioned above, wherein the mouthpiece [10] is shaped like a digital thermometer.

It is a still further objective of the invention to provide a probe [100] configured for measurement of vital signs mentioned above, wherein the mouthpiece [10] is shaped like a pacifier nipple.

It is a still further objective of the invention to provide a probe [100] configured for measurement of vital signs mentioned above, further comprising a protrusion [16] in front of the mouthpiece [10], wherein at such time as the mouthpiece is inserted in the mouth,
a. the protrusion [16] is configured be disposed outside the mouth and in contact with an upper lip of the mammalian subject; and
b. the protrusion [16] houses the one or more sensors, disposing the one or more sensors in the position suitable for measurement.

It is a still further objective of the invention to provide a probe [100] configured for measurement of vital signs mentioned above, further comprising a skirt [16′] in front of the mouthpiece [10], wherein, at such time as the mouthpiece is inserted in the mouth,
a. the skirt [16′] is configured be disposed outside the mouth, in contact with lips of the mammalian subject, and substantially covering the opening of the mouth; and
b. the skirt [16′] houses the one or more sensors, disposing the one or more sensors in the position suitable for measurement.

It is a still further objective of the invention to provide a probe [100] configured for measurement of vital signs mentioned above, further comprising at least one display [18] configured to display a result of any of the vital signs measurements.

It is a still further objective of the invention to provide a probe [100] configured for measurement of vital signs mentioned above, wherein the probe issues an audible signal upon display of the result.

It is a still further objective of the invention to provide a probe [100] configured for measurement of vital signs mentioned above, the color selected depending on the mammalian subject's condition as determined by results of measurement of the vital signs.

It is a still further objective of the invention to provide a probe [100] configured for measurement of vital signs mentioned above, further comprising a communication section [37] configured to communicate wireless data to an external device, wherein the wireless data comprises one or more results of any of the vital signs measurements.

It is a still further objective of the invention to provide a probe [100] configured for measurement respiratory rate mentioned above, further comprising a communication section [37] configured to communicate wireless data to an external device, the wireless data containing the waveform [FIG. 6] of physical changes.

It is a still further objective of the invention to provide a probe [100] configured for measurement of vital signs and comprising a communication section mentioned above, further configured to communicate with an external device via the internet.

It is a still further objective of the invention to provide a method for measuring a respiratory rate of a mammalian subject, comprising
a. placing at least one sensor near a nose of the mammalian subject;
b. measuring physical changes measured by the sensors;
c. analyzing a waveform of the physical changes to derive a frequency of the physical changes; and
d. determining the respiratory rate based on the waveform and frequency.

It is a still further objective of the invention to provide the method for measuring respiratory rate of mammalian subject mentioned above, wherein the placing at least one sensor near the nose of the mammalian subject is effected by inserting a mouthpiece of a probe into a mouth of the mammalian subject.

It is a still further objective of the invention to provide a method of measuring respiratory rate of a mammalian subject mentioned above, wherein determining the respiratory rate is made by equating the respiratory rate to the frequency.

It is a still further objective of the invention to provide a method of measuring respiratory rate of a mammalian subject mentioned above, wherein the measuring physical changes is measuring temperature.

It is a still further objective of the invention to provide a method of measuring respiratory rate of a mammalian subject mentioned above, wherein the analyzing is made using Fourier transforms.

It is a still further objective of the invention to provide a method of measuring respiratory rate of a mammalian subject mentioned above, further comprising measuring body temperature.

It is a still further objective of the invention to provide a method of measuring respiratory rate of a mammalian subject mentioned above, further comprising issuing an audible signal when respiratory rate has been determined.

It is a still further objective of the invention to provide a method of measuring respiratory rate of a mammalian subject mentioned above, further comprising transferring the measurement to an external device, the data containing the respiratory rate.

It is a still further objective of the invention to provide a method of measuring respiratory rate of a mammalian subject mentioned above, further comprising transferring data to an external device, the data containing the physical changes.

It is a still further objective of the invention to provide a method of measuring respiratory rate of a mammalian subject and comprising transferring data to an external device mentioned above, wherein a wireless connection is employed for transferring the data to the external device.

It is a still further objective of the invention to provide a method of measuring respiratory rate of a mammalian subject and comprising transferring data to an external device mentioned above, wherein the external device is a smartphone.

It is a still further objective of the invention to provide a method of measuring respiratory rate of a mammalian subject and comprising transferring data to an external device mentioned above, wherein an internet connection is employed for transferring the data to the external device.

It is a still further objective of the invention to provide the method of measuring respiratory rate of a mammalian subject and comprising transferring data to an external device employing a wireless connection mentioned above, wherein said wireless connection is implemented using a cellular connection.

It is a still further objective of the invention to provide the method of measuring respiratory rate of a mammalian subject and comprising transferring data to an external device employing a wireless connection mentioned above, wherein the wireless connection is implemented using any protocol in a group containing NFC, Wi-Fi, Bluetooth, and wireless USB.

It is a still further objective of the invention to provide a method for measurement of vital signs of a mammalian subject comprising steps of
a. Providing a probe [100] configured for measurement of the vital signs of a mammalian subject, the vital signs comprising at least one of respiratory rate, heart rate, and oxygen saturation of a mammalian subject, the probe comprising
   a mouthpiece [10] configured for insertion into a mouth of the mammalian subject; and
   one or more sensors configured to perform the measurement of vital signs, wherein the one or more sensors are each disposed in a position suitable for measurement when the mouthpiece is inserted in the mouth of the mammalian subject; and
b. operating the probe.

### BRIEF DESCRIPTION OF THE FIGURES

FIG. 1 is an external view of the probe according to some embodiments of the invention.

FIG. 2 is a mechanical sketch of the probe showing features inside the probe, according to some embodiments of the invention.

FIG. 3A is a frontal view of the probe (i.e. from a side of the probe facing the mammalian subject), according to some embodiments of the invention. FIG. 3B is a side view of the probe while in use by a mammalian subject.

FIG. 4A is a frontal view of the probe, according to some alternative embodiments of the invention. FIG. 4B is a side view of the probe while in use by a mammalian subject.

FIG. 5 shows a method for measuring vital signs of a mammalian subject, according to some embodiments of the invention.

FIG. 6 shows a method for measuring respiratory rate of a mammalian subject, according to some embodiments of the invention.

FIG. 7 shows a waveform of respiratory sensor reading measurements, collected and analyzed during measuring respiratory rate of a mammalian subject, according to some embodiments of the invention.

FIG. 8 shows a method of sharing vital-sign data with others and utilizing the vital-sign data, according to some embodiments of the invention.

FIG. 9A is a block diagram of sensors and electronic sections of a probe according to some embodiments of the invention; FIG. 9B is an exploded view showing a PCB of the probe.

### DETAILED DESCRIPTION

Overview

There is provided by the present invention a probe and method for measuring one or more vital signs of a mammalian subject. The mammalian subject can be a human being, especially including an infant or young child.

Means and methods for measuring temperature at the mouth are known, but the measurement of additional vital signs of a mammalian subject using a probe with a mouthpiece resembling a familiar oral thermometer is a novel concept. Key elements appearing in a probe according to one or more embodiments of the invention are as follows:
1. Incorporation of extra-oral sensors into the probe, deployed in the area of the mammalian subject's lip and near the nose when the mouthpiece of the apparatus is inserted (like a thermometer) into the mammalian subject's mouth. The mouthpiece is inserted and held in place in the mouth, and a stopper or protrusion helps to maintain correct positioning of the extra-oral sensors. This design approach enables the probe to measure additional vital signs, while, like a traditional oral thermometer, taking the mammalian subject's temperature using a temperature sensor in the mouthpiece.
2. One type of extra-oral sensors of the probe is placed close to the nostrils and detects a physical property changing with inspiration/expiration cycle of inhaled/exhaled air, enabling the mammalian subject's respiratory rate to be measured.
3. A reflective pulse oximetry sensor to measure heart rate and oxygen saturation is available, and the use of such a sensor on the forehead is well known; surprisingly it is herein shown that it is also possible to use such a sensor at the lip. It is a feature of the present invention to provide novel embodiments which deploy a reflective pulse oximetry sensor in the probe to take a pulse-oximetry reading.

The probe of the present invention is able to measure up to four vital signs simultaneously: body temperature, respiratory rate, heart rate and blood oxygenation, thereby enabling a new form of “thermometer” which also provides multiple vital signs measurements. The respiratory sensors and pulse oximetry sensor can be held in place by a part of the probe that, with the mouthpiece, further enhances positional stability of the extra-oral sensors, including but not limited to: 1) mounted on a protrusion extending from the apparatus, attached to the probe and seated against the mammalian subject's outer lip when the mouthpiece is inserted; 2) incorporated into a skirt of the mouthpiece, seated against the mammalian subject's outer lip and substantially covering the opening of the mammalian subject's mouth; 3) mounted on a sliding section which is pushed towards the lip once the mouthpiece is inserted into the mammalian subject's mouth; or 4) incorporated into a flexible arm which presses the probe against the lip.

In an exemplary embodiment of the invention, a probe for measuring vital signs of a mammalian subject includes at least one vital-sign sensor. A vital-sign sensor can be one or more of respiratory sensors for measuring respiratory rate and a pulse oximetry sensor for measuring heart rate and oxygen saturation. The vital-sign sensors are in mechanical connection with a mouthpiece of the probe. The mouthpiece can resemble a familiar, easy-to-use digital oral thermometer. When the mouthpiece is inserted in the mouth, the vital-sign sensor is disposed in a position suited for measurement, as described in detail herein. In some embodiments the probe also includes one or more other vital sign sensors, such as a body temperature sensor, which can be placed in the mouthpiece. Therefore, up to four vital signs—respiratory rate, heart rate, oxygen saturation, and body temperature—can be measured using this novel, easy-to-use probe.

In some exemplary embodiments, a probe includes at least one respiratory sensor in mechanical assembly with a mouthpiece. When the mouthpiece is inserted in the mouth, the respiratory sensors are disposed in a region near the mammalian subject's nose. The mouthpiece can be designed for positional stability of the probe, as described herein. A probe with respiratory sensors fixed to a mouthpiece held stably in the mouth can offer an advantage that the respiratory sensors remain correctly positioned when the mouthpiece is inserted in the mouth, facilitating respiratory rate measurement. The probe can offer an additional advantage that with the mouthpiece in the mouth, the mammalian subject is forced to breathe through the nose and not through the mouth, facilitating respiratory rate measurement. Both advantages are particularly beneficial when the mammalian subject is an infant or young child.

The respiratory sensors of the probe measure physical changes, near the nose, accompanying inhaling and exhaling of the mammalian subject. Some embodiments include two respiratory sensors, one disposed adjacent to each of the mammalian subject's two nostrils. A probe with a respiratory sensor disposed at each nostril can produce more reliable results. For example, if one nostril is partially clogged, the respiratory sensor adjacent to the other nostril will produce a better reading. A respiratory sensor of the probe can be a temperature sensor, such as a thermocouple, thermopile, or thermistor. Alternatively, a respiratory sensor can be a gas absorption sensor or airflow sensor.

In an exemplary embodiment of the invention, the probe includes a pulse oximetry sensor, such as a reflective pulse oximetry sensor, in mechanical connection with a mouthpiece. When the mouthpiece is inserted in the mouth, the pulse oximetry sensor is disposed in a region near the mammalian subject's lip. The pulse oximetry sensor disposed near the lip, in mechanical assembly with the mouthpiece, can offer an advantage that the pulse oximetry sensor remains correctly positioned when the mouthpiece is in the mouth, which can be particularly beneficial when the mammalian subject is an infant or young child.

In some embodiments of the invention, the mouthpiece is designed for positional stability of the probe when placed in the mammalian subject's mouth. For example, the mouthpiece can have a cross-sectional shape that is elongated horizontally along some length. The elongated shape can prevent the mouthpiece from rotating and the attached respiratory sensors and/or pulse oximetry sensor from moving out of position, thereby potentially enhancing measurement accuracy. In some embodiments, the mouthpiece is shaped like a digital oral thermometer, which can offer an additional advantage of measuring the mammalian subject's respiratory rate and/or heart rate and oxygen saturation as easily as taking his temperature with a traditional digital oral thermometer.

In some embodiments, the mouthpiece is shaped like a nipple of a pacifier, potentially improving ease of measurement and comfort to an infant mammalian subject.

In some embodiments of the invention, the probe includes a protrusion. The protrusion is positioned on the probe in front of the mouthpiece (i.e., positioned outside the mouth when the mouthpiece is inserted in the mouth). The protrusion can prevent the portion of the probe in front of the mouthpiece from entering the mouth, advantageously minimizing choking hazard, particularly if the mammalian subject is an infant or young child. The protrusion can be in contact with a lip of the mammalian subject, which can afford additional stability of the probe.

The protrusion can be shaped to conform to the lip or can be flexible, potentially enhancing anatomical conformity and mechanical stability of the probe and facilitating mammalian subject comfort.

In some embodiments, the protrusion houses the respiratory sensors. The protrusion is shaped and sized to dispose the respiratory sensors near the nose.

In some embodiments, the protrusion houses the pulse oximetry sensor. The protrusion includes an opening through which the pulse oximetry sensor can be in contact or close proximity with a lip of the mammalian subject.

In tandem with the mouthpiece, the protrusion can enhance probe stability, thereby facilitating reliable respiratory rate, heart rate, and oxygen saturation measurements.

In some embodiments of the invention, the mouthpiece of the probe further includes a body temperature sensor. The body temperature sensor can be located at a distal tip of the probe (i.e., a tip of the mouthpiece). The body temperature sensor can be inserted into the mouth, possibly under the tongue, in order to measure the mammalian subject's body temperature. A probe with a body temperature sensor can help further to diagnose or monitor the mammalian subject's medical condition.

In an exemplary embodiment of the invention, the method includes measuring a mammalian subject's vital signs. The method is implemented, for example, using a probe that is an embodiment of the invention.

In some embodiments, the method includes analyzing a waveform of physical changes near the mammalian subject's nose to determine frequency of physical changes and calculating the mammalian subject's respiratory rate; for example, by equating the respiratory rate to the frequency.

In some embodiments of the invention, the probe further includes one or more displays, such as LCDs, configured to display some or all measured vital signs.

In some embodiments, the probe is configured to issue an audible tone when one or more indicators have been determined.

In some embodiments, the probe further contains electronic circuitry configured for operation of the probe, such as the following: an analog front-end (AFE) section configured to read the sensors of the probe and provide digital outputs; a processor configured to read the digital outputs, perform the analysis described herein, and control the displays and communication section described herein; and a communication section configured to communicate with one or more external devices, thereby enabling transfer of measurement data to an external device, alerts and reporting of data to the user of an external device, and remote operation of the probe. The communication section may be configured to communicate with nearby devices, remote devices, or both. An example of an external device is a smartphone.

### DETAILED DESCRIPTION OF EMBODIMENTS

Non-limiting embodiments of the invention are now described in detail.

Probe

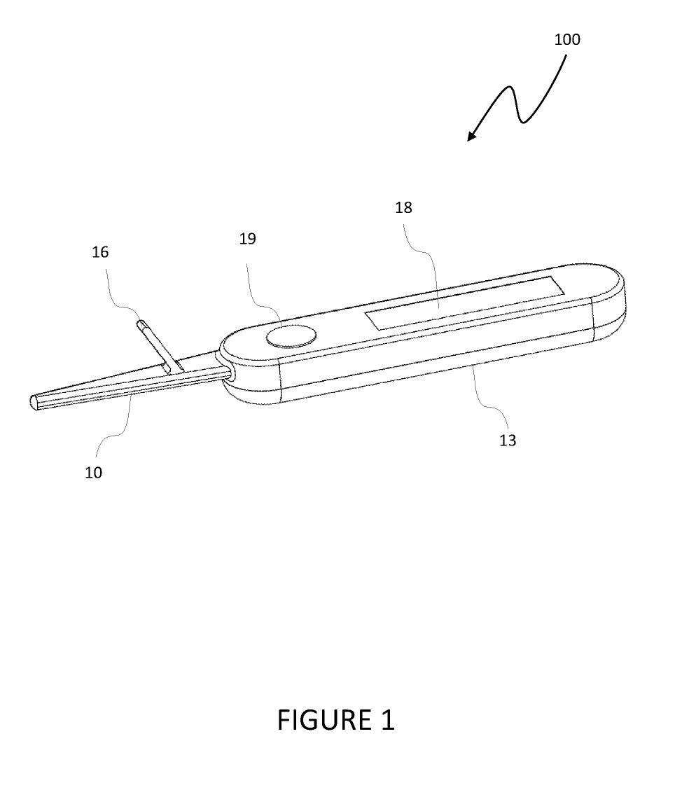

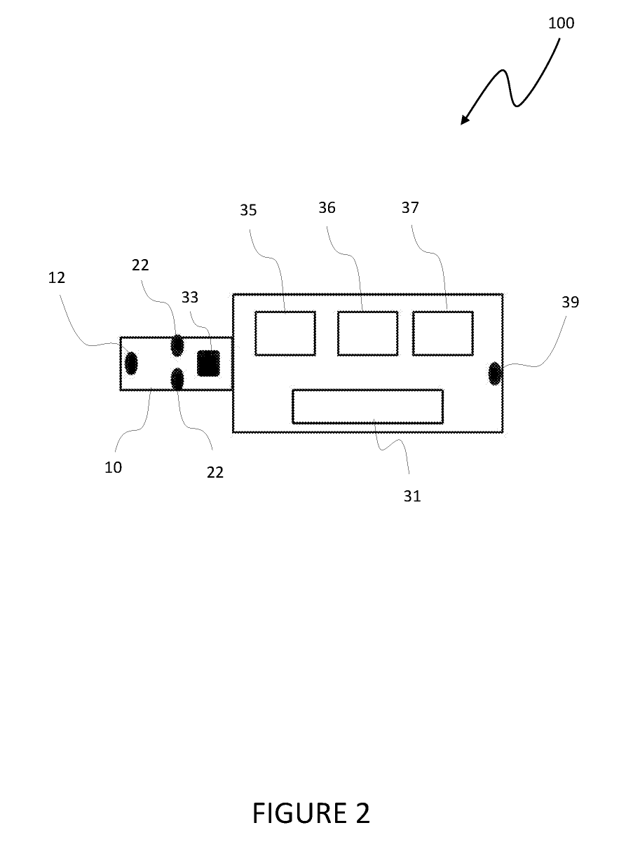

Reference is now made to FIG. 1, an external perspective view of an orally-inserted probe 100 according to some embodiments of the invention. Additionally, reference is made to FIG. 2, a schematic representation of the probe 100, revealing internal elements of the probe, according to some embodiments of the invention.

In an exemplary embodiment of the invention, a probe 100 for measuring respiratory rate of a mammalian subject includes one or more respiratory sensors 22—which can be housed in a protrusion 16 of probe 100—and a mouthpiece 10. Respiratory sensors 22 are connected to mouthpiece 10, either directly to mouthpiece 10 or by virtue of mutual assembly in probe 100, such that respiratory sensors 22 approach the nose when the mouthpiece 10 is inserted into a mouth of the mammalian subject. Probe 100 can include two respiratory sensors, each one positioned near one of the mammalian subject's nostrils. Use of two respiratory sensors positioned near each of the mammalian subject's nostrils can result in a more accurate measurement. For example, during a respiratory rate measurement described herein, if one respiratory sensor 22 gives a better S/N ratio (e.g., one of the mammalian subject's nostrils is partially clogged) can be used.

In some embodiments of the invention, respiratory sensors 22 are temperature sensors. Temperature sensors can be of low mass, allowing a temperature change of temperature sensor to occur within a short time between breaths of the mammalian subject. Temperature sensors can include one or more or thermocouples, thermopiles, or thermistors such as Murata NCP15WB473J03RC.

In some embodiments of the invention, respiratory sensors 22 of probe 100 are connected to an outer casing 13 of probe 100, arranged such that they will be oriented in a region 28 towards nostrils when mouthpiece 10 is inserted in mouth 26. Outer casing 13 can contain other features, described herein, of probe 100. In other embodiments, respiratory sensors are connected directly to mouthpiece 10. For example, probe 100 can be shaped like a pacifier, making the device smaller and potentially improving ease of measurement and comfort to an infant mammalian subject.

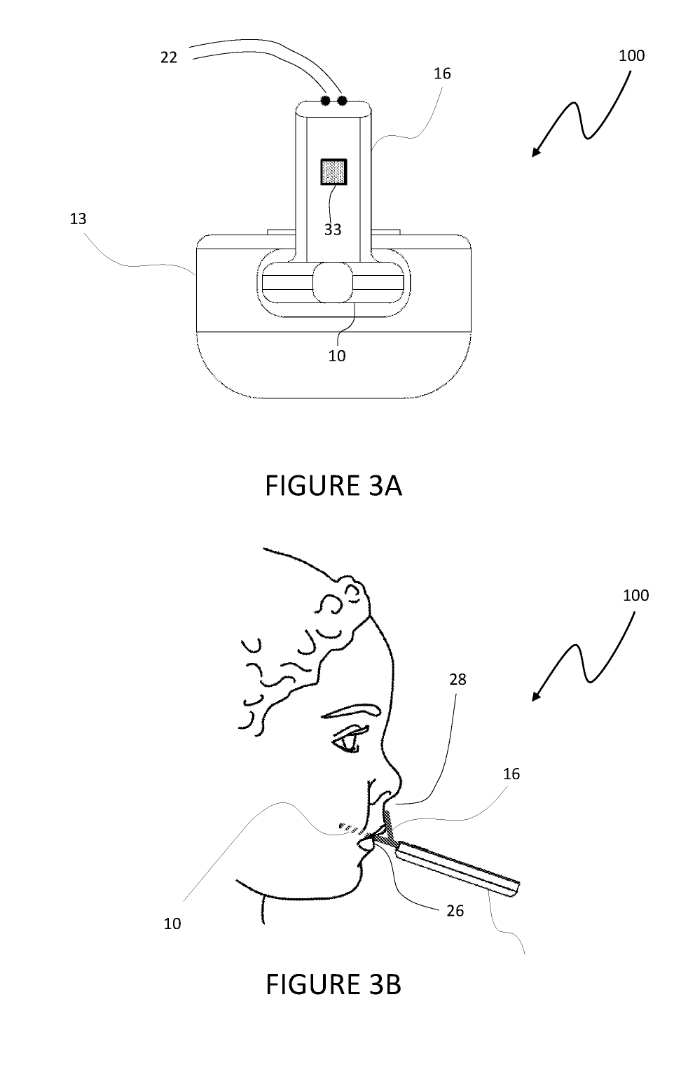

Reference is now also made to FIG. 3A, a front view of probe 100, and to FIG. 3B, showing probe 100 during measurement, according to some embodiments of the invention.

In some embodiments of the invention, respiratory sensors 22 are housed by a protrusion 16 of probe 100. Protrusion 16 is at a position on probe 100 and of a shape that disposes the respiratory sensors 22 in a region 28 near the mammalian subject's nostrils when mouthpiece 10 is inserted in the mouth 26. Protrusion 16 can be in contact with the mammalian subject's lip, which can improve positioning stability of respiratory sensors 22. Protrusion 16 of probe 100 can be slanted toward the mammalian subject when mouthpiece 10 is inserted in the mouth. Protrusion 16 can be flexible. The slanting and/or flexibility of protrusion 16 can offer anatomical conformity to the mammalian subject's upper lip, potentially providing physical comfort to the mammalian subject while using the probe 100 and also enhancing positional stability of respiratory sensors 22. Protrusion 16 can provide an obstruction to inserting the portion of probe 100 beyond mouthpiece 10 into the mouth, reducing choking hazard, particularly for infant and child mammalian subjects.

In an exemplary embodiment of the invention, a probe 100 for measuring a mammalian subject's heart rate and oxygen saturation (SpO₂) includes a pulse oximetry sensor 33 and a mouthpiece 10. In some embodiments, the pulse oximetry sensor 33 is a photo-reflective pulse oximetry sensor optimized for monitoring heart rate and SpO₂, such as the NJL5501R photo-reflector by New Japan Radio Co., Ltd. (Japan).

In some embodiments, pulse oximetry sensor 33 is housed by a protrusion 16 of probe 100. Protrusion 16 is at a position on probe 100 and of a shape that disposes pulse oximetry sensor 33 facing and proximate the mammalian subject's upper or lower lip when mouthpiece 10 is in the mouth. Conventionally, pulse oximetry measurements are made with a pulse oximetry sensor placed at a mammalian subject's finger or forehead. However, SpO₂ and heart rate measurements were surprisingly found to be equally accurate using a standard reflective pulse oximetry sensor placed near the mammalian subject's lip. In some embodiments, pulse oximetry sensor 33 is housed by a protrusion 16 of the probe 100. Protrusion 16 of probe 100 can be slanted toward the mammalian subject when mouthpiece 10 is inserted in the mouth. Protrusion 16 can be flexible. The slanting and/or flexibility of protrusion 16 can offer anatomical conformity to the mammalian subject's upper lip, potentially providing physical comfort to the mammalian subject while using the probe 100 and also enhancing positional stability of pulse oximetry sensor 33. Protrusion 16 can provide an obstruction to inserting the portion of probe 100 beyond mouthpiece 10 into the mouth, reducing choking hazard, particularly for infant and child mammalian subjects.

In some embodiments of the invention, the probe contains both respiratory sensors 22 and a pulse oximetry sensor 33. Both pulse oximetry sensor and respiratory sensors can be housed in a single protrusion 16 of probe 100. The protrusion 16 can offer advantages described herein.

In some embodiments of the invention, mouthpiece 10 of probe 100 is shaped to maintain positional stability of the probe 100. For example, some length of the mouthpiece 10 can be horizontally (i.e., direction of the longest dimension of the lips) elongated in cross-sectional shape. As an additional example, mouthpiece 10 can be shaped like a digital oral thermometer.

In some embodiments of the invention, mouthpiece 10 of probe 100 further contains a body temperature sensor 12. Body temperature sensor 12 can provide regular measurements of the mammalian subject's body temperature, providing a useful screening tool for diseases associated with increased body temperature, particularly infectious diseases such as pneumonia.

In some embodiments of the invention, probe 100 contains electronic sections. Electronic sections can include an analog front-end section 35, a processor 36, a communication section 37, and a power source 31. Electronic sections are described in further detail herein.

In some embodiments of the invention, probe 100 includes an external temperature sensor 39. External temperature sensor 39 can assist in performing internal device calibration and diagnostics of probe 100.

In some embodiments, probe 100 includes one or more of a display 18, to display values of measurements including one or more of respiratory rate, body temperature, oxygen saturation, and heart rate. Display 18 can be an LCD-type display. Display 18 can provide those treating the mammalian subject with immediate results without a requirement for communication between probe 100 and an external device. The display can include some form of color coding, such as red for tachypnea, which can be indicative of pneumonia.

In some embodiments, the probe 100 issues an audible signal when a measurement is complete. The audible signal can inform a caregiver that display 18 now shows the measured value of one or more vital signs. The audible signals can differ for different measured vital signs.

In some embodiments, probe 100 includes an on/off switch 19.

Additional sensors incorporated in the device 100 can include one or more pH sensors and/or enzymatic sensors.

Reference is also now made to FIG. 3A, a front view of probe 100, from an end including mouthpiece 10, according to some embodiments of the invention, and FIG. 3B, depicting the probe 100 while in use.

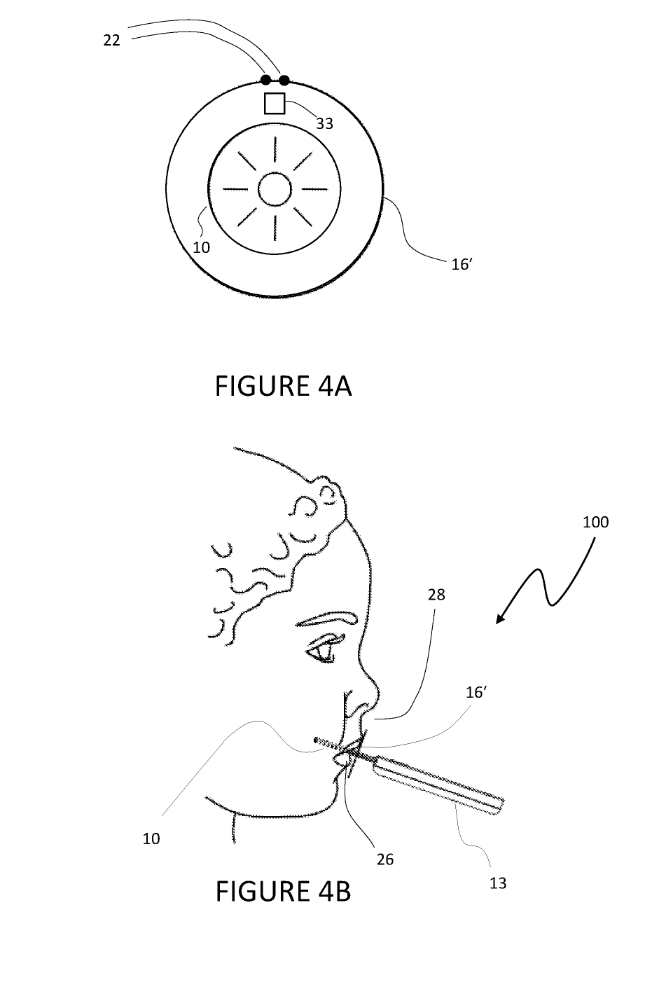

Reference is also now made to FIG. 4A, an end view of probe 100, from the end including mouthpiece 10, according to some alternative embodiments of the invention, and FIG. 4B, depicting the probe 100 while in use.

In some embodiments of the invention, probe 100 has a protrusion 16, described herein, that is slanted toward the mammalian subject when mouthpiece 10 is inserted in the mouth.

In some alternative embodiments of the invention, probe 100 has a skirt 16′, extended around the circumference of mouthpiece 10, that substantially surrounds the opening of the mouth 26 when mouthpiece 10 is inserted in the mouth of the mammalian subject. Skirt 16′ protrusion offers a relatively large surface area in contact with the mammalian subject around the mammalian subject's lips, which can improve positioning stability of respiratory sensors 22 and/or pulse oximetry sensor of the probe 100. In some embodiments, skirt 16′ is made of a soft material such as silicone, a gel, or latex rubber, which can enhance anatomical conformity and provide better positioning stability and comfort to the mammalian subject.

A skirt 16′ offers further potential advantages of preventing entrance into the mouth, of the probe beyond the mouthpiece (e.g., minimizing choking hazard) and covering the mouth around the lips so that the mammalian subject is forced to breathe only through his nose, thereby facilitating respiratory sensor measurements.

In some other alternative embodiments, the protrusion or skirt of FIGS. 3-4 is replaced by a sliding section pushed towards the lip once the mouthpiece is inserted in the mammalian subject's mouth, or incorporated into a flexible arm which presses the probe against the lip.

Measurement Methods

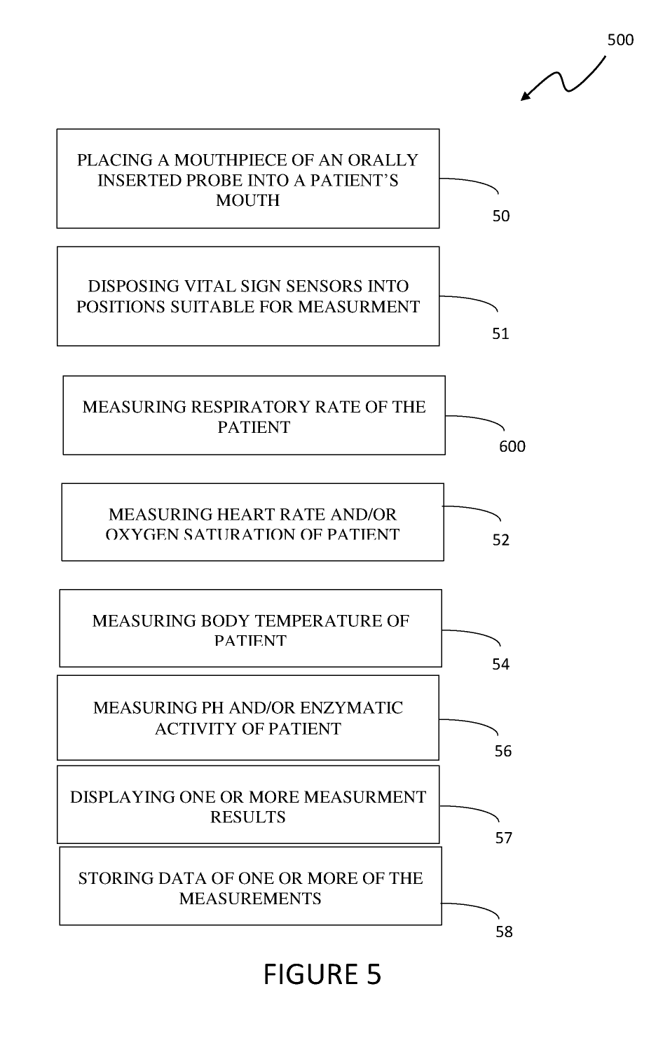

Reference is now made to FIG. 5, showing a method 500 for measuring vitals sign of a mammalian subject according, to some embodiments of the invention.

In an exemplary embodiment, method 500 includes placing a mouthpiece of an orally inserted vital signs probe, such as a probe that is an embodiment of the invention described herein, into a mammalian subject's mouth 50. Construction of the probe allows that placing the probe effects disposing vital signs sensors in a position suitable for measurement 51.

For example, disposing vital signs sensors 51 includes placing at least one respiratory sensor near a mammalian subject's nose. The mouthpiece and respiratory sensors are attached such that placing the mouthpiece 50 effects disposing the respiratory sensors near the mammalian subject's nose. Two respiratory sensors can be used, one near each nostril. The respiratory sensors can be temperature sensors. Using two respiratory sensors in proximity to each of the mammalian subject's nostrils can enhance an amplitude of a waveform, described herein, thereby improving the accuracy of the measurement.

Alternatively or additionally, for example, disposing vital signs sensors 51 includes placing a pulse oximetry sensor sensor adjacent to the mammalian subject's upper or lower lip. The mouthpiece and pulse oximetry sensor sensor are attached such that placing the mouthpiece 50 effects disposing the pulse oximetry sensor near the mammalian subject's upper or lower lip.

In an exemplary embodiment, method for measuring vital signs 500 includes measuring respiratory rate of the mammalian subject 600, described herein.

In an exemplary embodiment, method 500 includes measuring heart rate and/or oxygen saturation of the mammalian subject 52. For example, using a pulse oximetry sensor that is part of a probe that is an embodiment of the invention, described herein.

In some embodiments, method 500 includes measuring body temperature of the mammalian subject 54. For example, using a temperature sensor disposed within the mouthpiece of a probe, described herein.

In some embodiments, method 500 includes measuring pH and/or enzymatic activity of a mammalian subject 56.

In some embodiments, method 500 includes displaying one or more of the measurement results 57. Displaying 57 can be on one or more displays, described herein, of a probe that is an embodiment of the invention.

In some embodiments, method 500 includes storing data of one or more of the vital signs measurements in memory 59. The memory can be memory, described herein, of a probe that is an embodiment of the invention. A possible advantage of storing data is better enabling transferring the data to an external device, as described herein. If the data are respiratory sensor readings, storing the data in memory can offer an advantage of enabling a processor of a probe that is an embodiment of the invention to analyze a waveform of respiratory sensor readings and determine respiratory rate, as described herein.

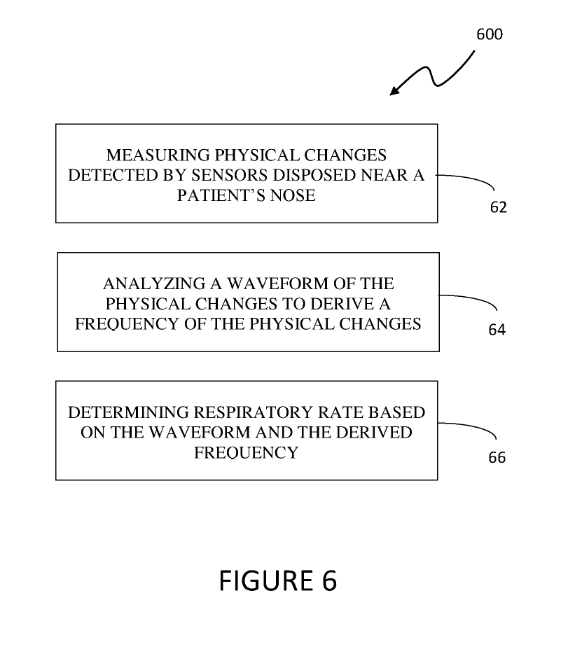

Reference is now made to FIG. 6, showing a method 600 of measuring respiratory rate of a mammalian subject, according to some embodiments of the invention.

In an exemplary embodiment of the invention, method 600 includes measuring physical changes detected by respiratory sensors disposed near a mammalian subject's nose 62. The physical changes can be caused by the inhaling and exhaling of the mammalian subject. The physical quantity measured can be temperature, or can include, alternatively or in addition, one or more of another quantity, such infrared absorption or air flow. A measurement can be made by comparing the outputs of the respiratory sensors, and selecting the output with strongest signal strength. Selection of the strongest output from several sensors can allow for a more accurate respiratory rate measurement compared with using the output of a single sensor. This is especially advantageous given when comparing the outputs of two sensors, each measuring near a different one of two nostrils of the mammalian subject, as there is always a nostril giving a stronger signal and they tend to alternate every few hours.

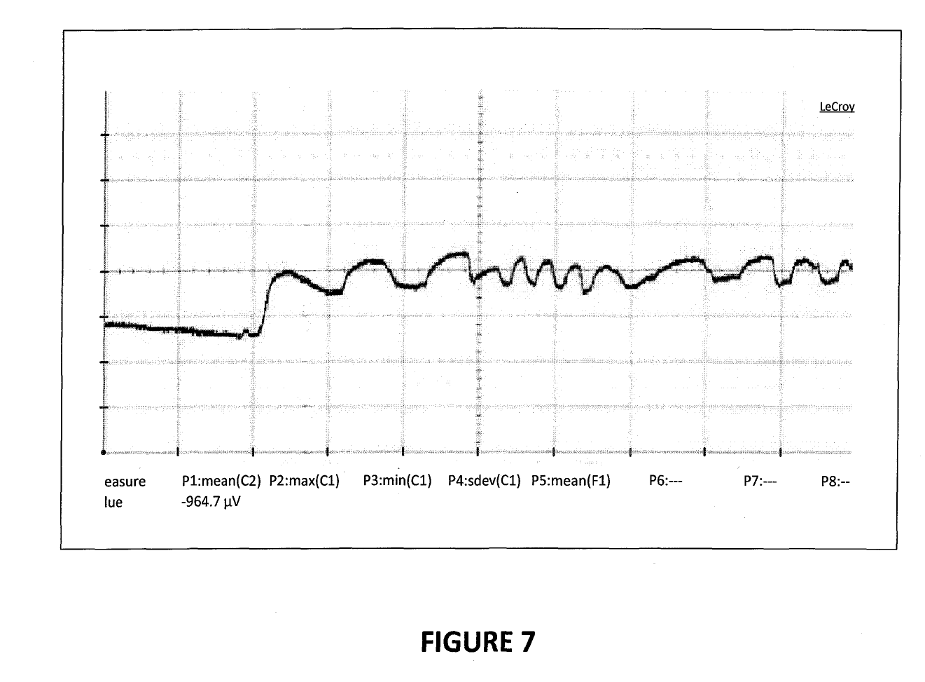

Method 600 further includes analyzing a waveform of the physical changes detected by the sensors, such as the waveform shown in FIG. 7, to derive a frequency of the physical changes 64. Analyzing 64 can be implemented by a processor of a probe that is an embodiment of the invention, described herein. Alternatively, analyzing 64 can be implemented by an external device, described herein. Analyzing 64 can include using any suitable method, such as computing the Fourier transform of the waveform or an interval of the waveform.

Method 600 further includes determining respiratory rate based on the waveform and the derived frequency 66. For example, the respiratory rate is set equal to the derived frequency.

Sharing and Utilizing of Data

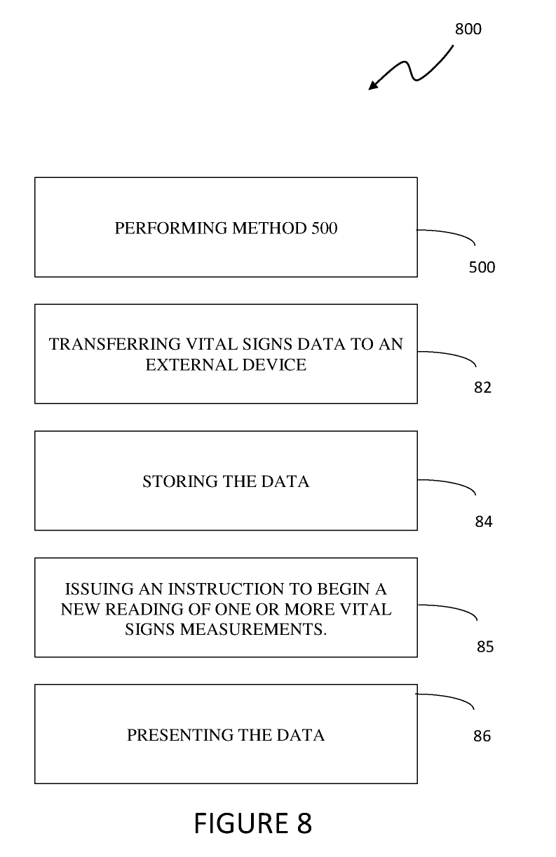

Reference is now made to FIG. 8, showing a method 800 of sharing vital signs data with an external device such as a PC, tablet, wearable device or smartphone and of utilizing the data, according to some embodiments of the invention.

Method 800 includes performing method 500, measuring vital signs of a mammalian subject, described herein. Method 800 further includes transferring respiratory rate data to an external device 82. Transferring 82 can be initiated by a processor, descried herein, that is an embodiment of the invention. Transferring 82 may be implemented using a protocol for wireless RF communication. The protocol can be NFC, Wi-Fi, Bluetooth, wireless USB, or other protocols known in the art. Alternatively, or in addition, the protocol can be a cellular technology—which can enable transferring 82 where a nearby device is unavailable—such as GSM, 3G, 4G, or other protocols known in the art. Transmitting data 82 may be implemented, entirely or in part, through a network, such as a LAN, a WAN, the Internet, or a VPN. The external device can be a smartphone, a laptop PC, a router, a web server, or any device with either wired or wireless communication ability. The external device can be located either nearby the mammalian subject or at a remote location. The external device can be in possession, for example, of the mammalian subject, a nearby caregiver, or a remotely located doctor or other medical personnel. Transmitting data can be implemented automatically upon proximity between the probe and external device. Transmitted data may further be retransmitted to a third device; for example, a web server.

The transferred data of step 82 can include respiratory rate measurements. Alternatively, the data can be raw data points of the waveform. Transmitting raw data can enable the step of analyzing the waveform 64 to be performed on the external device or any other device receiving the raw data, alleviating a Processor of the probe, described herein, of this function. Analysis by an external device instead of the probe can, among other advantages, reduce power consumption of the probe. Transmitted data can also include measurements of body temperature, oxygen saturation, and pulse rate. In some embodiments, an application on the external device, or any other device receiving the transmitted data, displays one or more of the measurements, which can allow medical personnel to monitor measurements in real time.

In some embodiments, method 800 further includes storing the data 84. Storing 84 can be on the external device, or on any other device that may receive the data; for example, a server containing a database of mammalian subject medical records. Storing the data 84 allows, for example, archiving measurements for later viewing a history and historical trends of the mammalian subject's condition, described herein.

In some embodiments, an external device can issue an instruction to begin a new reading of one or more vital signs measurements 84. The ability of an external device to issue an instruction to begin a reading allows software developers more flexibility in writing applications implementing methods for sharing and utilization of vital-signs measurements, embodiments of methods described herein.

In some embodiments, method 800 further includes presenting the data 86. Presenting the data 86 can be in the form of alerting a person via the external device or any other device with access to the data. For example, an application on a smartphone of the mammalian subject can alert the mammalian subject of a potentially significant change in the respiratory rate. For further example, a sudden change in body temperature or respiratory rate can cause an alarm to issue on a smartphone or computer of medical personnel, potentially allowing immediate attention and response to the condition.

Presenting the data 86 can entail, for example, an application with access to archived data presenting a history of mammalian subject respiratory rate. A physician can, for example, compare the history and trends at different periods of the mammalian subject's treatment.

Electronics

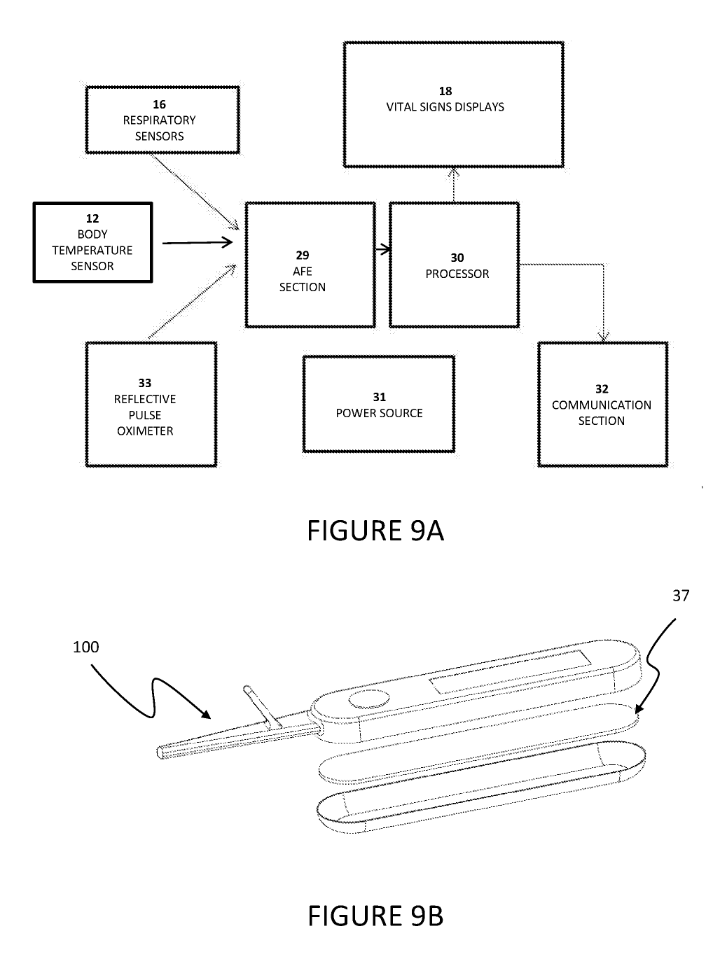

Reference is now made to FIG. 9A, a block diagram of probe 100 according to some embodiments of the invention, revealing the electronic features of the probe and their interoperability with other features of the probe.

In some embodiments, probe 100 includes a power source 31. Power source 31 can be one or more disposable batteries, such as AA, AAA, coin cell, or button cell batteries. Alternatively, power source can be a rechargeable battery, such as a lithium-ion battery.

In some embodiments, probe 100 includes an AFE section 35 that receives the analog outputs of the vital signs sensors, such as respiratory, pulse oximetry, and body temperature sensors. AFE section 35 contains circuitry for reading and digitizing each sensor output. Circuitry can include, a pre-amplifier, signal conditioning circuit, and A/D converter. The AFE section provides digitized sensor readings, for input into a processor.

In some embodiments, probe 100 contains a processor 36. Processor 36 receives digital outputs from AFE section 35. Processor 36 is configured to control functioning of the probe 100. In some embodiments, processor 36 is configured to perform computations, such as those required for analysis of a respiratory sensor reading waveform, whose method, described herein, is an embodiment of the invention. The processor can be implemented using a CPU, FPGA, or any other information processing components known in the art. In some embodiments, processor 36 contains memory to which the digitized sensor readings and/or computational results can be stored. The memory can be RAM, flash memory, or any other types of memory components known in the art. There can be more than one type of memory, for example serving different purposes such as containing program instructions, short-term data storage, and long-term data storage. Memory is useful, for example, for transmitting data in bulk, e.g., packets, during transfer of respiratory sensor readings from probe 100 to an external device, embodiments described herein.

In some embodiments, probe 100 contains a communication section 37. Communication section 37 is responsible for implementing communicating data from the probe 100 to an external device. The communicating of data is one feature of a method of sharing and utilizing vital signs data, described herein, that is an embodiment of the invention. The communication section contains the circuitry and components needed for wireless communication, configured to operate according to one or more protocols such as NFC, Wi-Fi, Bluetooth, or wireless USB.

Some functions of communication section 37 can be controlled by processor 36 and other functions of communication section performed controlled internally.

Referring now to FIG. 9B; there is shown a circuit board 37 that can contain one or more of electronic sections: power source 31, AFE section 35, Processor 36, and communication section 32. The circuit board 37 can be single sided or double sided and can contain multiple layers. The circuit board 37 may in fact comprise several smaller circuit boards, which can facilitate manufacture and test of the electronic sections.

It is understood that the above electronics sections are conceptual only. Some described functions of one or more of the sections may be implemented, in whole or in part, using common components.

1. A probe [100] configured for measurement of vital signs of a mammalian subject, said vital signs comprising at least one of respiratory rate, heart rate, and oxygen saturation of a mammalian subject, comprising
a. a mouthpiece [10] configured for insertion into a mouth of said mammalian subject; and
b. one or more sensors configured to perform said measurement of vital signs,
wherein said one or more sensors are each disposed in a position suitable for measurement when the mouthpiece is inserted in the mouth of the mammalian subject.

2. The probe [100] according to claim 1, wherein
a. said vital signs include respiratory rate and said one or more sensors comprise one or more respiratory sensors; and
b. said position suitable for measurement is in proximity to a nose of the mammalian subject, thereby wherein said one or more respiratory sensors are disposed in proximity to the nose of the mammalian subject at such time as said mouthpiece is in the mouth of the mammalian subject,
wherein by means of a waveform of physical changes measurable by said at least one respiratory sensor the respiratory rate of the mammalian subject is determinable.

3. The probe [100] according to claim 1, wherein
a. said vital signs include at least one of heart rate and oxygen saturation and said one or more sensors comprise a reflective pulse oximetry sensor; and
b. said position suitable for measurement is adjacent to and facing a lip of the mammalian subject, thereby wherein said reflective pulse oximetry sensor is disposed adjacent to and facing said lip of the mammalian subject at such time as said mouthpiece is in the mouth of the mammalian subject.

4. The probe according to claim 1, wherein
a. said vital signs further include body temperature; and
b. said mouthpiece [10] further comprises a body temperature sensor [24], disposed in said mouthpiece [10] and configured for measurement of body temperature.

5. The probe according to claim 2, wherein
a. said at least one respiratory sensor comprises two respiratory sensors;
c. the two respiratory sensors are in proximity to two nostrils of the mammalian subject, one sensor in proximity with each nostril.

6. The probe [100] according to claim 2 or claim 5, wherein said at least one respiratory sensor [16] comprises one or more temperature sensors.

7. The probe according to claim 5, wherein said one or more temperature sensors are selected from a group including thermocouples thermopiles, and thermistors.

8. The probe according to claim 2 or any of claim 5-claim 7, further comprising a processor [36] configured to perform an analysis of said waveform [FIG. 4] generated by said at least one respiratory sensor [16], said processor [36] configured to determine the respiratory rate.

9. The probe according to claim 8, wherein the processor [36] is further configured to perform said analysis using a Fourier transform of the waveform.

10. The probe according to any of claim 2-claim 4, wherein a cross section of at least a portion of said mouthpiece [10] is horizontally elongated.

11. The probe according any of claim 2-claim 4, wherein said mouthpiece [10] is shaped like a digital thermometer.

12. The probe according to any of claim 2-claim 4, wherein said mouthpiece [10] is shaped like a pacifier nipple.

13. The probe of claim 2 or claim 3, further comprising a protrusion [16] in front of the mouthpiece [10], wherein at such time as the mouthpiece is inserted in the mouth,
a. said protrusion [16] is configured be disposed outside the mouth and in contact with an upper lip of the mammalian subject; and
b. said protrusion [16] houses said one or more sensors, disposing said one or more sensors in said position suitable for measurement.

14. The probe of claim 2 or claim 3, further comprising a skirt [16′] in front of the mouthpiece [10], wherein, at such time as the mouthpiece is inserted in the mouth,
a. said skirt [16′] is configured be disposed outside the mouth, in contact with lips of the mammalian subject, and substantially covering the opening of the mouth; and
b. said skirt [16′] houses said one or more sensors, disposing said one or more sensors in said position suitable for measurement.

15. The probe according to any of claim 2-claim 4, further comprising at least one display [18] configured to display a result of any of said vital signs measurements.

16. The probe according to claim 15, wherein the probe issues an audible signal upon display of said result.

17. The probe according to any of claim 2-claim 4 or to claim 15, further comprising a color display configured to display a color, said color selected depending on the mammalian subject's condition as determined by results of measurement of said vital signs.

18. The probe according to any of claim 2 to claim 4, to claim 8, or to claim 9, further comprising a communication section [37] configured to communicate wireless data to an external device, wherein said wireless data comprises one or more results of any of said vital signs measurements.

19. The probe according to claim 2, further comprising a communication section [37] configured to communicate wireless data to an external device, said wireless data containing said waveform [FIG. 6] of physical changes.

20. The probe according to claim 18 or claim 19, further configured to communicate with an external device via the internet.

21. A method for measuring a respiratory rate of a mammalian subject, comprising
a. placing at least one sensor near a nose of the mammalian subject;
b. measuring physical changes measured by the sensors;
c. analyzing a waveform of the physical changes to derive a frequency of said physical changes; and
d. determining the respiratory rate based on said waveform and frequency.

22. The method according to claim 21, wherein said placing at least one sensor near the nose of the mammalian subject is effected by inserting a mouthpiece of a probe into a mouth of said mammalian subject.

23. The method according claim 21, wherein said determining is made by equating the respiratory rate to said frequency.

24. The method according to claim 21, wherein said measuring physical changes is measuring temperature.

25. The method according to claim 21, wherein said analyzing is made using Fourier transforms.

26. The method according to claim 21, further comprising measuring body temperature.

27. The method according to claim 21, further comprising issuing an audible signal when respiratory rate has been determined.

28. The method according to claim 21, further comprising transferring said measurement to an external device, said data containing said respiratory rate.

29. The method according to claim 21, further comprising transferring data to an external device, said data containing said physical changes.

30. The method according to claim 28 or claim 29, wherein a wireless connection is employed for transferring said data to said external device.

31. The method according to any of claims 28-claim 30, wherein said external device is a smartphone.

32. The method according to claim 28-claim 30, wherein an internet connection is employed for transferring said data to said external device.

33. The method according any of claim 30, wherein said wireless connection is implemented using a cellular connection.

34. The method according to claim 30, wherein said wireless connection is implemented using any protocol in a group containing NFC, Wi-Fi, Bluetooth, and wireless USB.

35. A method for measurement of vital signs of a mammalian subject comprising steps of
a. Providing a probe [100] configured for measurement of said vital signs of a mammalian subject, said vital signs comprising at least one of respiratory rate, heart rate, and oxygen saturation of a mammalian subject, said probe comprising
a mouthpiece [10] configured for insertion into a mouth of said mammalian subject; and
one or more sensors configured to perform said measurement of vital signs,
wherein said one or more sensors are each disposed in a position suitable for measurement when the mouthpiece is inserted in the mouth of the mammalian subject; and
b. operating said probe.
